(19) United States
(12) Patent Application Publication
(10) Pub. No.: US 2023/0107339 A1
(43) Pub. Date: Apr. 6, 2023

(54) MESHED DERMAL TISSUE MATRIX PRODUCTS

(71) Applicant: LifeCell Corporation, Madison, NJ (US)

(72) Inventors: Hui Xu, Plainsboro, NJ (US); Sangwook Park, Virginia Beach, VA (US); Melissa Richter Bowley, Newport, RI (US)

(21) Appl. No.: 17/843,321

(22) Filed: Jun. 17, 2022

Related U.S. Application Data
(63) Continuation of application No. 16/782,478, filed on Feb. 5, 2020, now abandoned.
(60) Provisional application No. 62/801,723, filed on Feb. 6, 2019.

(51) Int. Cl. A61L 27/36; A61L 27/50; A61F 2/12; A61B 17/06; A61F 2/00; A61N 1/362

(52) U.S. Cl. CPC A61L 27/362; A61L 27/50; A61F 2/12; A61B 17/06166; A61L 27/3633; A61F 2002/0081; A61N 1/362; A61F 2002/0068; A61L 2430/34; A61F 2220/0008; A61L 2430/04; A61F 2210/0057

(57) ABSTRACT

The present disclosure provides meshed acellular dermal tissue matrix compositions, devices, and methods of use. The meshed devices can be used in conjunction with a variety of implants such as breast implants or tissue expanders.

This application is a continuation of U.S. patent application Ser. No. 16/782,478, filed Feb. 5, 2020, which claims priority under 35 U.S.C. § 119 to U.S. Provisional Application No. 62/801,723, which was filed on Feb. 6, 2019 and is hereby incorporated by reference in its entirety.

The present disclosure relates to tissue matrix products. In particular, embodiments of the invention relate to meshed acellular tissue matrix products, including methods of formation and use in either clinical or research settings.

Soft tissue, such as skin, fascia, muscle, and adipose tissue, is ubiquitous throughout the body. Permanent loss of soft tissue, which is often disfiguring and debilitating, can result from any number of causes. Examples include trauma, infection, vascular compromise, ionizing radiation, and resection of malignancy, to name a few. Generally, collagen-based soft tissue, including skin, fascia, adipose tissue in the breast, and overlying muscle and other structures in various locations, does not regenerate when lost. Consequently, surgical procedures have been developed to replace such lost soft tissue. These procedures historically have included translocation of autologous soft tissue, such as placement of skin grafts, local rotation flaps, including fasciocutaneous and myocutaneous flaps, vascular pedicle-based “free flaps,” the use of temporary tissue expanders to stretch and expand autologous tissue to fill or cover a defect, placement of synthetic or natural tissue-based implants, and related applications. Other procedures employ related techniques to augment existing soft tissue contours for aesthetic reasons, including but not limited to augmentation of the breast and buttocks in women and the pectoral and deltoid regions in men, for example.

Acellular dermal matrix (“ADM”) compositions derived from human and animal dermis, such as ALLODERM® and STRATTICE® produced by LIFECELL® CORPORATION (Madison, N.J.)), are widely used in aesthetic and reconstructive surgical procedures. Such materials provide a number of advantages and can be used to replace or augment soft tissue structures.

ADM products, although versatile, may be difficult to apply smoothly and with uniform tension over the underlying anatomic structures or curved prostheses, such as a synthetic breast implant, tissue expander, pacemaker, or other implantable device.

Accordingly, there is a need for ADM products formed to conform to the shape of tissue implants and surrounding anatomic structures without wrinkling, deformation of overlying skin and surrounding tissue, or to more evenly distribute tension created when these products are surgically anchored to underlying tissues.

The present disclosure provides meshed acellular tissue matrix compositions, devices, and methods of use.

Accordingly, in some embodiments, a soft tissue reconstruction product comprising an acellular dermal matrix formed as a generally flat sheet, having slits extending through a thickness of the flat sheet, is provided. The slits form a first mesh configuration comprising a regular pattern of slits with a length:length ratio and a length:width ratio.

Also provided is a method of treating a soft tissue, comprising identifying an anatomic site within a soft tissue; selecting a soft tissue treatment device comprising a meshed acellular tissue matrix, wherein the meshed acellular tissue matrix comprises a generally flexible sheet having slits extending through a thickness of the sheet, the sheet further having a top surface, a bottom surface, and a peripheral border; implanting the treatment device in or proximate the soft tissue; and securing at least a portion of the treatment device tissue in or near the soft tissue. In some embodiments, the soft tissue is a breast.

Also provided is a meshed acellular dermal matrix, comprising a generally flexible sheet, having slits extending through a thickness of the flat sheet; wherein the sheet is a substantially flexible sheet having a top surface, a bottom surface, and a peripheral border. The meshed tissue matrix can be used in conjunction with an implant. In some embodiments, the soft tissue implant is a synthetic implant. In some embodiments, the implant is a tissue expander. In some embodiments, the implant is a breast implant. In some embodiments, the implant, is surgically implanted in a sub-muscular position, a subcutaneous position, or a mixed sub-muscular and subcutaneous position.

### BRIEF DESCRIPTION OF THE DRAWINGS

Reference will now be made to exemplary embodiments, examples of which are illustrated in the accompanying drawings. Wherever possible, the same reference numbers will be used throughout the drawings to refer to the same or like parts. The drawings are not necessarily to scale.

FIGS. 1a-d are diagrams of four example mesh configurations;

FIG. 2 is a chart listing dimensions of four example mesh configurations;

FIGS. 3a-d are photomicrographs of tissue at an ADM-implant interface stained to show grades of smooth muscle cell actin (SMC-actin) staining intensity;

FIGS. 4a-e are photographs demonstrating a primate breast reconstruction model using meshed ADM product;

FIG. 5 is a bar graph comparing SMC-actin staining intensity at an implant-ADM interface with four example mesh configurations, unmeshed ADM product, and a control skeletal muscle-synthetic implant interface;

FIG. 6 is an exemplary illustration of an implant being enveloped by the mesh configuration;

FIG. 7 is an exemplary illustration of calf implants being enveloped by the mesh configuration;

FIG. 8 is an exemplary illustration of a pacemaker being enveloped by the mesh configuration; and

FIG. 9 is an exemplary illustration of a chemotherapy port implant being enveloped by the mesh configuration.

### DETAILED DESCRIPTION OF EXEMPLARY EMBODIMENTS

Reference will now be made in detail to various embodiments of the disclosed products, devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including,” and other forms, such as “includes” and “included,” is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure relates generally to meshed tissue matrix products for surgical soft tissue reconstruction or augmentation procedures, and systems and methods relating to such products. The products can be used for tissue augmentation, repair or regeneration of damaged tissue, and/or correction of tissue defects. The products can also or alternatively be used to cover or envelope implantable devices such as pacemakers, infusion pumps, or the like.

The use of ADM, however, may be complicated by the contours of underlying anatomic structures or implants. Convex and concave curves intersect in complex ways, depending on the anatomic region and the particular individual. For example, the anterior chest wall is externally convex, transitioning to concave at the lateral border of the pectoralis major muscle, convex at the lateral chest wall and then changing again to concave in the axilla. Meshed tissue products can be more easily adapted to cover or conform to these various shapes and curvatures. As such, the products, devices, systems, and methods discussed herein can be suitable for a wide range of surgical applications addressing replacement, repair or augmentation of soft tissue in many anatomic locations.

Use of ADM products in soft-tissue treatment procedures wherein synthetic implants or tissue expanders are implanted has been shown to be useful in mitigating or preventing pericapsular inflammation that often leads to formation of a dense, fibrous capsule around implants comprised of synthetic materials. A meshed ADM product has advantages over a non-meshed ADM product for certain uses. The meshed ADM product allows for even distribution of tension across multiple curved surfaces, versus a non-meshed product. Interstices of the mesh allow for the drainage of fluid to facilitate resorption of blood or serous fluid which often accumulates around a synthetic soft tissue implant, such as a breast implant. Enhanced drainage facilitates resorption of such fluid and reduces the risk for infection, deformity, and migration or disruption of the implant. Also, a meshed product can be “expanded,” wherein a pattern of holes is created when tension is applied non-parallel to, for example, perpendicular to a long axis of slits in the mesh. Mesh expansion, consequently, allows for greater surface area coverage using the same amount of meshed ADM product versus a non-meshed sheet of ADM.

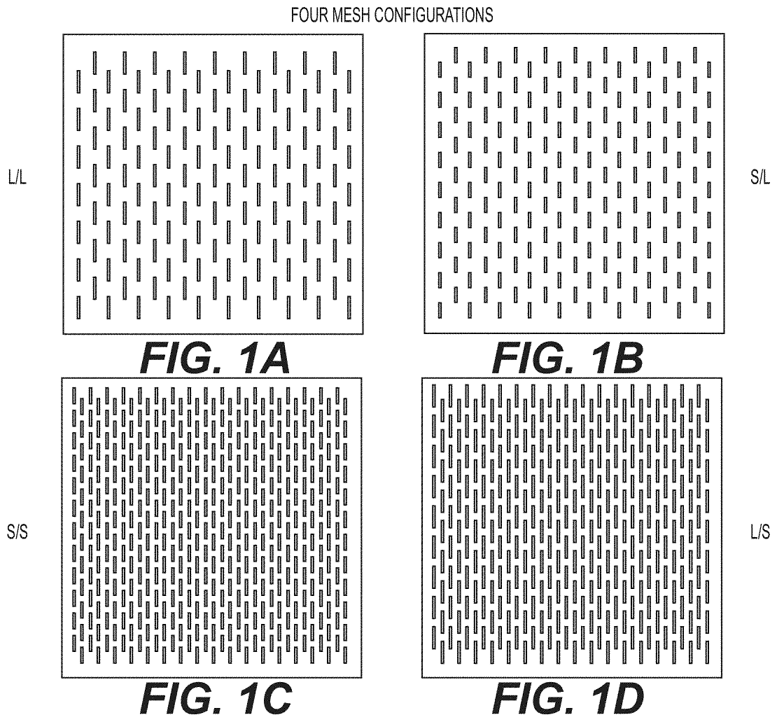

FIGS. 1a-d are diagrams of four exemplary mesh configurations. As shown in FIGS. 1a-d, cuts are made in the ADM product to allow for lengthening or shortening along a dimension. For the purpose of disclosures recited herein, a length:length ratio means the ratio of a slit length to the distance between slits along a long axis of the slits. A “length:width” ratio means the ratio of the slit length to the distance between slits across (generally perpendicular to) the long axis of the slits. FIG. 1a shows a meshed ADM product “L/L” having linear slits at a length:length ratio of about 1.5:1 and a length:width ratio of about 1.25:1. FIG. 1B shows a meshed ADM product “S/L” having linear slits at a length:length ratio of about 1.0:1 and a length:width ratio of about 1.0:1. FIG. 1c shows a meshed ADM product “S/S” having linear slits at a length:length ratio of about 2.0:1 and a length:width ratio of about 2.0:1. FIG. 1d shows a meshed ADM product “L/S” having linear slits at a length:length ratio of about 3.0:1 and a length:width ratio of about 3.0:1.

In some embodiments, the meshed ADM product is provided with a single mesh configuration. In some embodiments, the meshed ADM product is provided with two mesh configurations. In some embodiments, the meshed ADM product is provided with greater than two mesh configurations.

In some embodiments, a first mesh configuration has a length:length ratio of 1:1, or 1.5:1, or 2:1, or 2.5:1, or 3:1, or 3.5:1, or 4:1, or greater than 4:1. In some embodiments, a second mesh configuration has a length:width ratio of 1:1, or 1.5:1, or 2:1, or 2.5:1, or 3:1, or 3.5:1, or 4:1, or greater than 4:1.

In an example embodiment, linear slits are cut into an ADM product generally parallel to each other and staggered in position relative to one another. This is by way of example and not meant to be limiting. There are many possible clinical and research applications utilizing a meshed ADM product, therefore, many possible sizes, shapes, and arrangements of slits are possible in a meshed ADM product. For example, in some embodiments, the mesh slits are oriented generally orthogonal to a long axis of the ADM product, including in the examples shown in FIGS. 1a-d. In some embodiments, the mesh slits are curved slits oriented generally parallel to an edge of the ADM product. In some embodiments, the mesh slits are curved slits oriented generally other than parallel to the edge of the ADM product. In some embodiments, the slits are holes having the same or similar shapes. In some embodiments, the slits are holes having different shapes. In some embodiments, the slits are holes having about the same sizes. In some embodiments, the slits are holes having different sizes. In some embodiments, the slits have about the same length:length ratios, as shown by FIGS. 1a-d. In some embodiments, the slits have different length:length ratios (not shown). In some embodiments, the slits have the same length:width ratios, as shown by FIGS. 1a-d. In some embodiments, the slits have different length:width ratios (not shown). In some embodiments, the slits or holes are arranged in a repeating pattern, such as the example embodiments shown in FIGS. 1a-d. In some embodiments, the slits are present across substantially the entire area of the ADM product. In some embodiments, the slits are present in a first area of the ADM product but are not present in a second are of the ADM product (not shown in the drawing figures).

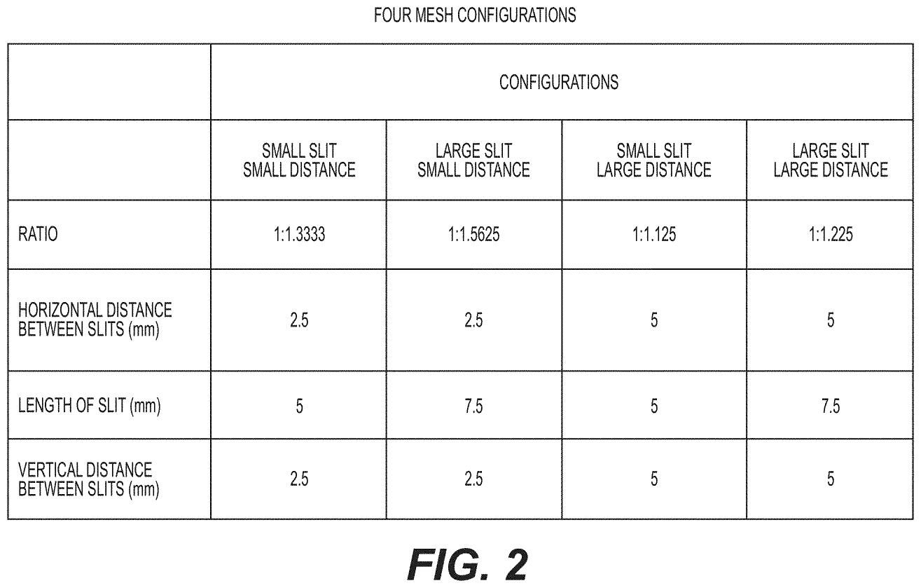

FIG. 2 is a chart listing dimensions of four exemplary mesh configurations. FIG. 2 shows four example mesh configurations, which are designated “small slit small distance,” “large slit small distance,” “small slit large distance,” and “large slit large distance,” corresponding to the S/S, L/S, S/L, and L/L designations of FIGS. 1a-d. FIG. 2 also shows additional parameters defining an example ADM mesh configuration, including a ratio (length:length ratio), a horizontal distance between slits, a slit length, and a vertical distance between slits.

In some embodiments wherein the meshed ADM product comprises slits in a regular pattern, the meshed ADM product is partially or fully “expanded” during a surgical implantation procedure by applying a tension to the ADM. The ADM product is then applied to cover or retain a tissue implant or anatomic structure having an externally convex surface. In other applications, the meshed ADM tissue product is left unexpanded or partially expanded to cover or retain a tissue implant or anatomic structure having an externally concave surface. In still other applications, the meshed ADM tissue product is applied to cover or retain a tissue implant or anatomic structure having an external concave surface adjacent to and transitioning with an external convex surface. In locations where the ADM product covers an external convex surface, the slits can be expanded, creating larger spaces in the meshed ADM. In locations where the ADM product covers an external concave surface, however, the cuts in the ADM product may close, either partially or fully, in some embodiments, to allow the product to more easily fully contact the concave surface without redundancy and without causing wrinkling or buckling of the product. Additionally, different sized openings between bridging segments of ADM between the slits tend to normalize tension across the ADM. For example, tension of the meshed ADM product is reduced across an underlying convex surface whereupon the meshed ADM product is not pulled/elevated off of an adjacent underlying concave surface.

In some embodiments, the meshed ADM product is used as a retaining device. The meshed ADM product may be cut to a specific size and shape particular to a specific application. For example, a flexible sheet of the meshed ADM product may be cut into an elongated, curved shape specific to define the lateral and inframammary boundaries of an implant used to reconstruct or augment a female breast. In some embodiments, a flexible sheet of the meshed ADM product is manufactured in a pre-cut or pre-formed two-dimensional shape wherein additional further cutting may be performed in the operating room by a plastic or other reconstructive surgeon. Many shapes, sizes, and customization options are possible when forming meshed ADM products.

In some embodiments, the ADM can assist in retaining an acellular tissue matrix implant, for example, a shaped or three-dimensional tissue matrix implant. The acellular tissue matrix implant is an adipose-tissue derived implant, a dermal-derived implant, a muscle-derived implant, a cartilage-derived implant, a bone-derived implant, or a composite derived from two or more tissue types, in some embodiments. In some embodiments, the structure retained is a synthetic implant, such as a prosthesis such as a breast implant. The synthetic implant may, alternatively, be a tissue expander, such as a silicone tissue expander, for example, a tissue expander for use in two stage breast reconstruction procedures. Other possible implants or tissue may be retained by the meshed ADM product, including infusion pumps, pacemakers, defibrillators, shunts, cardiac assist devices, to name a few.

In some embodiments, the meshed ADM product is provided as a composite tissue product. For example, the meshed ADM product may comprise a meshed ADM sheet coupled to a second acellular tissue matrix, in sheet or other form. In some embodiments, for example, the meshed ADM product is a meshed ADM sheet coupled to a tissue matrix having a formed, three-dimensional shape. The sheet can be a flexible material having a top surface, a bottom surface, and a peripheral border, in some embodiments. The peripheral border may comprise at least two edges, including a first edge having a substantially curved, linear, or mixed configuration. The second edge, in some embodiments, has a second configuration. As discussed further herein, the meshed ADM can form part of a treatment system, including a breast implant, a tissue expander, or other tissue implant. The meshed ADM may be formed as a shaped ADM product consistent with the disclosures found in U.S. Pat. No. 8,986,377, and Patent Publication Nos. US 2018/0055624 and US 2017/0071725, the disclosures of which are included entirely herein by reference.

As noted, the meshed ADM product and related devices discussed herein can be used for treatment of a breast. Accordingly, the meshed ADM product can be part of a system for treating a breast. In some embodiments, the system comprises a sheet of meshed ADM product and an implant, such as a breast implant or breast tissue expander. A variety of suitable implants (e.g., saline filled breast implants) and tissue expanders are used, according to the embodiment.

The tissue matrices used to produce the devices described herein can include a variety of different materials. For example, an acellular tissue matrix or other tissue product can be selected to allow tissue ingrowth and remodeling to assist in regeneration of tissue normally found at the site where the matrix is implanted. An acellular tissue matrix, when implanted on or into subdermal tissue, fascia, mammary tissue, or other tissue, may be selected to allow regeneration of the tissue without excessive fibrosis or scar formation. In certain embodiments, the devices can be formed from ALLODERM® or STRATTICE™ (LIFECELL® CORPORATION, Madison, N.J.) which are human and porcine acellular dermal matrices, respectively. Alternatively, other suitable acellular tissue matrices can be used. For example, a number of biological scaffold materials as described by Badylak et al., or any other similar materials, can be used to produce tissues with a stable three-dimensional shape. Badylak et al., “Extracellular Matrix as a Biological Scaffold Material: Structure and Function,” Acta Biomaterialia (2008), doi:10.1016/j.actbio.2008.09.013. The devices described herein can be produced from a variety of different human or animal tissues including human, porcine, ovine, bovine, or other animal tissues.

In some cases, the meshed ADM product is produced from materials that include a basement membrane on at least one surface. For example, the devices can be produced from an acellular dermal matrix, and either the top surface or bottom surface can include an epithelial basement membrane across the surface. During implantation, the meshed ADM having a basement membrane should generally be positioned such that the basement membrane surface is positioned facing away from the most vascular tissue. For example, as discussed below, when implanted next to a breast implant or tissue expander, the basement membrane covered surface may face towards the implant or tissue expander such that the surface not including a basement membrane faces overlying vascularized tissue.

Meshed ADM products, devices, and related systems disclosed herein can have other shapes and configurations. For example, the meshed ADM product, in some embodiments is coupled to a pre-shaped three-dimensional acellular tissue matrix. In some embodiments, the pre-shaped three-dimensional tissue matrix is an acellular dermal matrix. In some embodiments, the pre-shaped three-dimensional tissue matrix is an acellular adipose tissue matrix. In some embodiments, the pre-shaped three-dimensional tissue matrix is an acellular muscle tissue matrix.

In some embodiments, the meshed ADM product includes a sheet of meshed acellular tissue matrix. The sheet of acellular tissue matrix comprises a flexible sheet with a top surface and a bottom surface (not shown in the drawing figures). The meshed ADM product also includes a peripheral border, wherein the peripheral border comprises a first edge having a first shape, and a second edge having a second shape (not shown). In some embodiments, the first shape, the second shape, or the first shape and the second shape are linear. In some embodiments, the first shape is a first curve. In some embodiments, the second shape is a second curve. The disclosures herein intend to include any combination of linear and/or curved shaped sheet-like meshed ADM products, without limitation.

Also disclosed herein are methods for treating a breast. An example method comprises steps for implantation of a system for surgical breast procedures, including a pre-shaped tissue matrix implanted with a breast implant or tissue-expander, according to certain embodiments. The method can first include identifying an anatomic site within a breast. (As used herein, “within a breast” will be understood to be within mammary tissue, or within or near tissue surrounding the breast such as tissue just below, lateral or medial to the breast, or beneath or within surrounding tissues including, for example, under chest (pectoralis) muscle, and will also include implantation in a site in which part or all of the breast has already been removed via a surgical procedure). The site can include, for example, any suitable site needing reconstruction, repair, augmentation, or treatment. Such sites may include sites in which surgical procedures (mastectomy, lumpectomy, debridement) have been performed, sites where aesthetic procedures are performed (augmentation or revision of augmentation), or sites needing treatment due to other causes, including disease or trauma.

After selection of the site, a treatment device is selected. As noted above, various devices including acellular tissue matrices can be used, and the devices can include a meshed ADM in the form of a flexible sheet having a top surface, a bottom surface, and a peripheral border. The peripheral border and shape of the devices can include any configuration discussed herein.

The method can also include securing at least a portion of the meshed ADM product or device to a patient. For example, in one embodiment, a portion of the device is secured to a chest wall, to surrounding fascia, or to part of an implant or a tissue expander. In one embodiment, at least a portion of an edge of the ADM is secured to tissue using, for example, suture, or other suitable attachments. In addition, other portions of the device, including portions of an edge of the ADM, can be secured to tissue, or if appropriate to the implant to tissue expander (e.g., via surface features on a tissue expander).

The methods disclosed herein can also include implantation of an implant or a tissue expander under or near part of the meshed ADM product or device. In some cases, no implant or expander will be used, but the meshed ADM product is implanted to provide added tissue, e.g., for incision closure after mastectomy. In some cases, the implant or expander is implanted at the same time as the meshed ADM product, or in a subsequent surgical procedure.

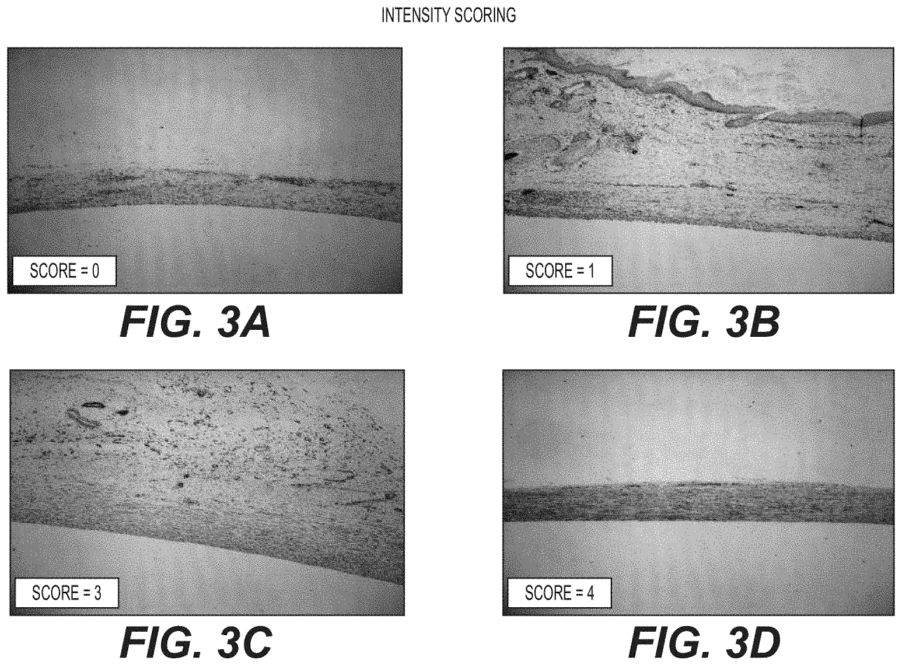

Tissue matrix was tested in a primate model with silicone implant. A scoring system was developed, which is discussed with respect to FIGS. 3a-d. FIGS. 3a-d are photomicrographs of tissue at an ADM-implant interface stained to show grades of smooth muscle cell actin (SMC-actin) staining intensity. As discussed herein, ADM products are known to mitigate formation of a dense, fibrous capsule which often forms around a synthetic implant, such as a breast implant or a soft tissue expander. It is not known, however, whether a meshed ADM product allows migration of myoepithelial cells through interstices created by open slits, leading to formation of a fibrous capsule. Accordingly, the inventors created a scoring system to rate the intensity of capsule formation. The scoring system rates formation of a fibrous capsule from “0” to “4,” as shown by FIGS. 3a-d. The inventors chose to omit “2,” leaving this designation for possible future assignment to describe a tissue SMC-actin staining intensity between a score of 1 and 3. A score of “0” reflects no detectable staining of SMC-actin, and is shown in FIG. 3a. A score of “1” reflects minimally detectable staining of SMC-actin, and is shown in FIG. 3b. A score of “3” reflects moderate-high staining of SMC-actin, and is shown in FIG. 3c. A score of “4” reflects dense staining of SMC-actin, and is shown in FIG. 3d. Accordingly, FIG. 3a is a photomicrograph showing a cross-sectional view of a soft tissue boundary including ADM with no fibrosis and no observable SMC-actin staining. FIG. 3b is a photomicrograph showing a similar cross-sectional view of a soft tissue-implant boundary with mild fibrosis. FIG. 3c is a photomicrograph showing a similar cross-sectional view of a soft tissue-implant boundary with moderate fibrosis. FIG. 3c is a photomicrograph showing a similar cross-sectional view of a soft tissue-implant boundary with marked fibrosis typical of a fibrous capsule.

In applications wherein the meshed ADM product is employed to cover and retain a synthetic implant, therefore, the size of interstices should be adequate to distribute tension across a dimension of the ADM product and to allow for drainage of fluid from around the implant. The interstice size should not be so large, however, as to compromise ADM mitigation of fibrous capsule formation, and thus allowing formation of a fibrous capsule, or continuous capsule-like tissue growth, adjacent or around the synthetic implant.

In one example, and in some other embodiments, a meshed ADM product is used to retain a breast implant surgically positioned partially or completely beneath a pectoralis major muscle of a patient, wherein the meshed ADM product is anchored, using sutures, stables, or other surgical anchors and techniques known in the art, along a lateral border of the pectoralis major muscle extending along an inferior border of the pectoralis major muscle, to fix the implant in a stable position and to resist rotation or migration of the implant from beneath the pectoralis major muscle. The meshed ADM may cover all external surfaces of the implant to prevent or mitigate formation of a peri-implant fibrous capsule while permitting drainage of fluid from around the implant through the slits. Alternatively, the meshed ADM product may cover only a portion of the external surface of the implant, such as an anterior surface, an anterior-inferior-lateral surface extending from beneath the pectoralis major muscle, or a posterior surface adjacent to the chest wall. Indeed, many configurations are possible, depending on the surgical application, disease-specific and patient-specific considerations, the type of material comprising the implant, the anatomic location of the implant, and, possibly, other factors.

In some embodiments, the meshed ADM product is inserted into a human patient, an animal patient, or a laboratory animal without an implant. For example, the meshed ADM product may be used in the surgical repair of hernia, such as an abdominal wall hernia, a sliding esophageal hernia, a paraesophageal hernia, a diaphragmatic hernia, or other internal hernia. In some embodiments, the meshed ADM product is implanted across a tissue defect, such as a defect in a muscle fascia, a dura, a cortical bone, a mucosa, a cartilage, or other defect of in soft tissue, cartilage, or bone.

### Example: Implantation of Meshed pADM in a Primate Model

Porcine acellular dermal matrix was formed with mesh configurations and implanted along with a silicone tissue expander.

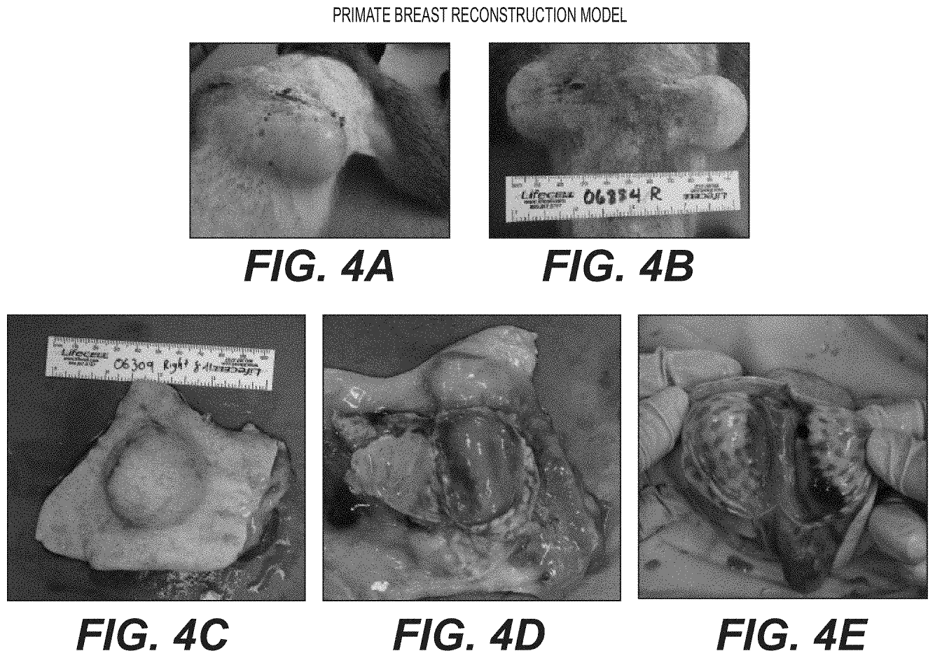

FIGS. 4a-e are photographs demonstrating a primate breast reconstruction model using meshed ADM product. FIGS. 4a-e show implantation of a silicone ball in the subcutaneous space on the back of a laboratory animal (primate). On one side, the ball is surgically placed in the subcutaneous space in a paraspinous or posterolateral location on the back of the animal. A sheet of ADM meshed in a 2:1 length-length and 2:1 length:width ratio is draped over the implant and sutured and the skin is closed over the meshed ADM product. FIG. 4a shows a postoperative photograph showing the surgical wound on the animal's back shortly after closure of the incision. FIG. 4b shows a photograph of the same region after complete healing, approximately ten (10) weeks later. Ten (10) weeks after implantation, the animal is sacrificed, the implant is excised, and the peri-implant soft tissue is examined. FIG. 4c shows a block of excised tissue comprising the incorporated meshed ADM product, the silicone ball, and underlying soft tissue after removal at necropsy. FIG. 4d shows the implant cavity following incision of the cavity and removal of the silicone ball. The shiny surface of a fibrous capsule is clearly visible where the silicone ball contacted soft tissue of the body wall without an intervening meshed ADM product. A cut edge of the meshed ADM product is visible around the perimeter of the opened implant cavity. FIG. 4e shows the incorporated meshed ADM.

The results of staining for SMC-actin positive marker of fibrotic capsule, similar to the primate study depicted by FIGS. 4a-e, suggest that both human and porcine ADM prevent capsule formation in a similar way, by reducing or preventing infiltration of the pericapsular space with myofibroblast cells. Although myofibroblast cells were present within the mesh interstices, no continuous capsule-like tissue or growth of tissue extending through and outside of the interstices was observed in any of the four (4) mesh configurations. This suggests that meshed porcine ADM product prevents a continuous fibrous capsule or scar tissue formation between the mesh and the expander, at least in the mesh configurations tested. In the interstices, the myofibroblast cell layer is thinner and the SMC-actin staining is weaker than the capsule above the muscle, further supporting the conclusion that porcine ADM product prevents capsule formation, even when meshed. Although the configuration with large spaces and large distance between spaces (L/L) demonstrated occasional significant inflammation, there is no evidence to suggest that the interstices were the source of the inflammation, because the same interstices with less myoepithelial tissue present did not show this response.

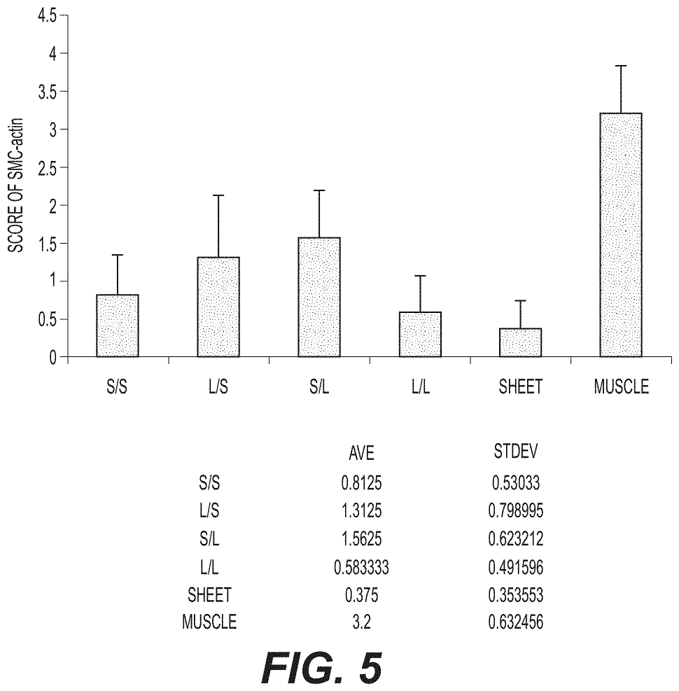

FIG. 5 is a bar graph comparing SMC-actin staining intensity at an implant-ADM interface with four example mesh configurations, unmeshed ADM product, and a control skeletal muscle-synthetic implant interface. Taken together, the data support the use of meshed ADM products for breast and other soft-tissue reconstruction procedures. Notably, given the present data, the ability to expand a meshed ADM product while retaining the anti-capsule formation effect of the ADM is an unexpected and surprising result. As shown by FIG. 5, all four mesh configurations tested demonstrated a slight or greater intensity of SMC-actin staining than use of an un-meshed ADM product sheet and significantly less intensity than with no ADM product, shown in the “muscle” bar. Regardless, it remains expected that there is a ratio wherein larger interstices resulting from larger mesh ratios and broader expansion of the meshed ADM product tensioned over an externally convex surface of a synthetic implant or tissue expander will result in myofibroblast infiltration of the interstices and capsular growth. At this and larger ratios, is it anticipated this important advantage of using ADM to surround a synthetic implant may be diminished, although this is not yet known at the time of this disclosure.

On preliminary mechanical testing measuring the tensile strength of meshed versus unmeshed ADM product, the meshed product failed at a lower applied tensile force. Regardless, however, the meshed ADM product exceeded tolerances for tensile strength established for manufacture of the unmeshed product. The preliminary testing of the meshed ADM product suggests that tensile strength of a sheet of meshed ADM product is not critically compromised by meshing.

In another example, and in some other embodiments, a meshed ADM product is used to retain a synthetic implant. The meshed ADM may cover all external surfaces of the implant to prevent or mitigate formation of a peri-implant fibrous capsule while permitting drainage of fluid from around the implant through the slits. Alternatively, the meshed ADM product may only cover a portion of the external surface of the implant, such as an anterior surface. Indeed, many configurations are possible, depending on the surgical application, disease-specific and patient-specific considerations, the type of material comprising the implant, the anatomic location of the implant, and, possibly, other factors.

As discussed above, the implant that is covered by the meshed ADM may be a synthetic implant used in cosmetic procedures. In one embodiment, the synthetic implant is a calf implant. The meshed ADM can provide greater structural support for the calf implant by expanding around the implant to cover more surface area of the implant. In some embodiments, the meshed ADM only partially covers a synthetic implant. The slits of the meshed ADM provide proper drainage of fluid and help the surrounding tissue more easily grow around a synthetic implant.

In another embodiment, the implant that is covered by the meshed ADM is a synthetic implant for use in various surgical procedures. An example of a synthetic implant used is a pacemaker. By covering the implant with the meshed ADM the surrounding tissue is less likely to scar or form surrounding capsule. This would help the patient have fewer or reduced complications when being treated with a pacemaker.

In another embodiment, the synthetic device that is covered by the meshed ADM is a chemotherapy port. This type of device is more rigid and is harder for tissue to support. By covering a synthetic devices with the meshed ADM, the surrounding tissue is less likely to scar or develop surrounding capsule. In addition, chemotherapy ports tend to leave an indent or hole in the insertion site. By covering the port with the meshed ADM the insertion site after treatment is more likely to grow back lost tissue or to allow easier removal without remaining scar tissue.

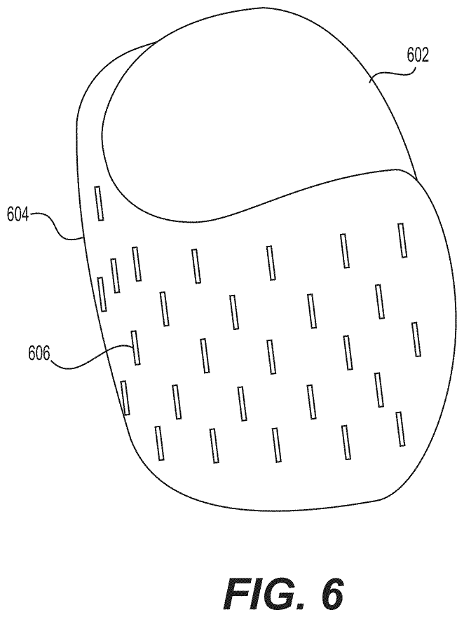

FIG. 6 is an exemplary embodiment of the meshed tissue matrix 604 enveloping a synthetic implant 602. The slits 606 allow the meshed tissue matrix 604 to expand around the synthetic implant 602, covering more surface area than an unmeshed tissue matrix would be able to. As shown in FIG. 6, the meshed tissue matrix 604 is flexible and can be curved or wrapped around a synthetic implant due to the slits 606.

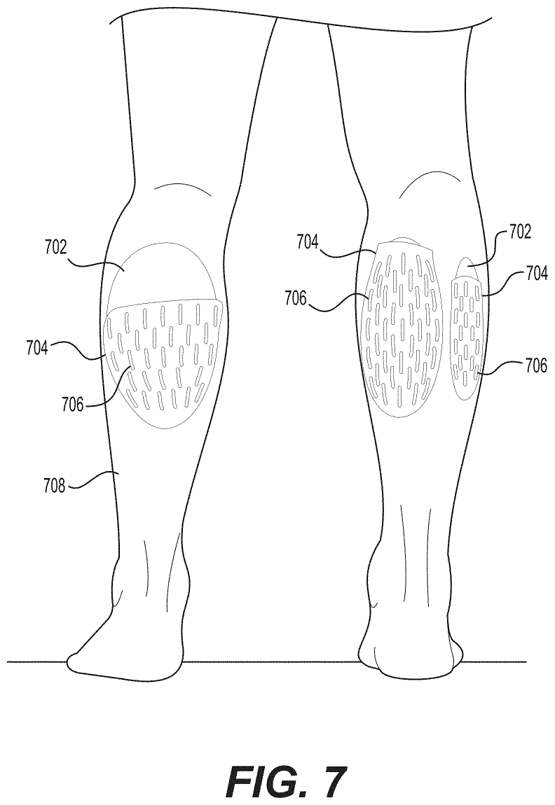

FIG. 7 is an exemplary embodiment of the meshed tissue matrix 704 enveloping calf implants 702 in a human calf 708. The meshed tissue matrix 704 has slits 706 that allow the meshed tissue matrix to expand around the calf implants 702. In one embodiment of FIG. 7, the meshed tissue matrix 704 partially envelopes the calf implants 702. In another embodiment of FIG. 7, there are multiple meshed tissue matrices 704 enveloping or partially enveloping multiple calf implants 702. As shown in FIG. 7, the meshed tissue matrix can be various shapes and sizes to accommodate the form of a synthetic implant, notably a calf implant.

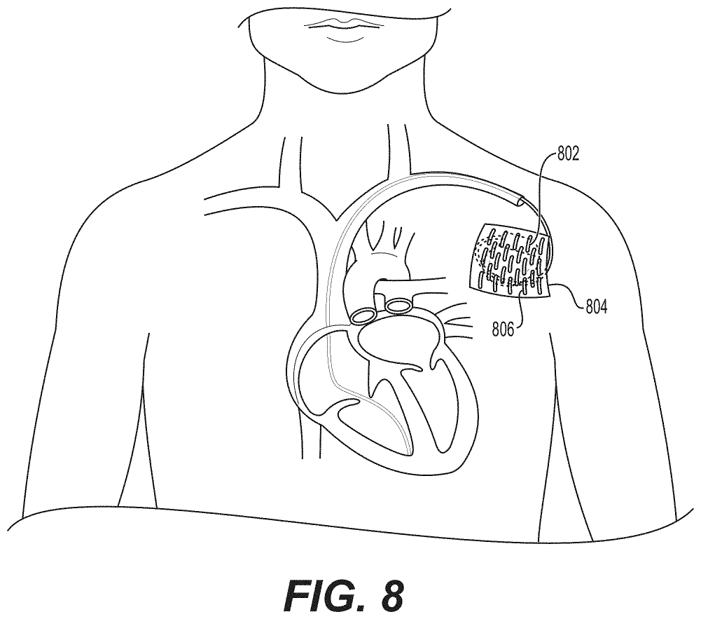

FIG. 8 is an exemplary embodiment of the meshed tissue matrix 804 enveloping a pacemaker 802. The meshed tissue matrix 804 has slits 806 that allow the meshed tissue matrix 804 to expand around the pacemaker 802. The slits 806 can help prevent the formation of scar tissue around the pacemaker 802, as well as allow proper drainage of fluids around the pacemaker 802.

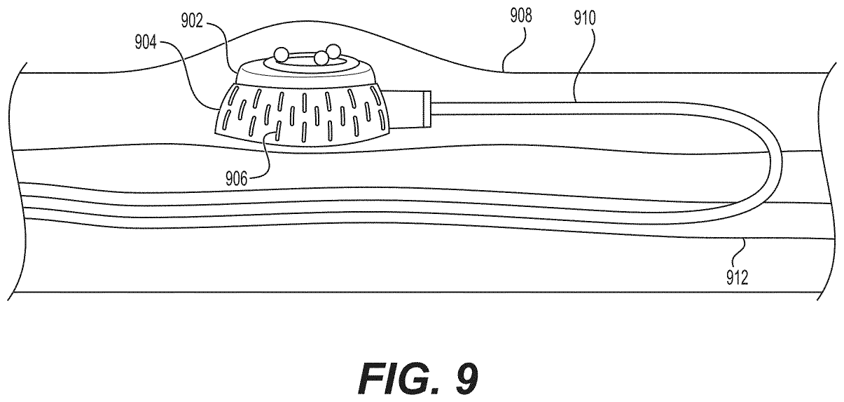

FIG. 9 is an exemplary embodiment of the meshed tissue matrix 904 surrounding a chemotherapy port 902. The chemotherapy port 902 is placed under the skin 908. The tube 910 is directed into the blood vessel 912. The meshed tissue matrix 904 has slits 906 that allow the meshed tissue matrix 904 to expand around the chemotherapy port 902 to more fully envelope the chemotherapy port 902. The slits 906 can help prevent the formation of scar tissue and allow for proper drainage of fluids around the chemotherapy port 902. One of ordinary skill in the art would be aware that in chemotherapy patients, it is not uncommon to have a small indent or hole in the area of the chemotherapy port insertion site after treatment. By placing the meshed tissue matrix 904 around a chemotherapy port during initial insertion, the surrounding tissue will be able to form faster and with less scar tissue after the chemotherapy port is removed.

The embodiments and examples set forth herein are presented to explain aspects of the present inventions and practical application, and to thereby enable those of ordinary skill in the art to make and use the inventions. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, without departing from the spirit and scope of the forthcoming claims.

What is claimed is:

1. A tissue product, comprising:
an acellular dermal matrix formed as an expandable sheet comprising a plurality of slits having a long axis, the slits extending through a thickness of the sheet and arranged in a pattern forming a mesh configuration;
wherein the mesh configuration has a first predetermined ratio of a length of each slit to a distance between slits along a long axis of the slits and a second predetermined ratio of a width of each slit to the distance of each slit across the long axis of the slits; and
wherein the mesh configuration allows for regenerative tissue ingrowth while reducing scar tissue formation.

2. The tissue product of claim 1, wherein the first predetermined ratio of a length of each slit to a distance between slits along a long axis of the slits is less than about 1:1, or about 1:1, 1.5:1, 2:1, 2.5:1, 3:1, 3.5:1, 4:1, or greater than about 4:1.

3. The tissue product of claim 1, wherein the second predetermined ratio of a width of each slit to the distance between each slit across the long axis of the slits is less than about 1:1, or about 1:1, 1.5:1, 2:1, 2.5:1, 3:1, 3.5:1, 4:1, or greater than about 4:1.

4. The tissue product of claim 1, wherein the mesh configuration distributes tension across a curved surface when applied to a soft tissue.

5. The tissue product of claim 1, wherein the mesh configuration allows for the drainage of fluid to facilitate resorption of blood or serous fluid.

6. The tissue product of claim 1, wherein the mesh configuration allows for expansion to provide greater surface area coverage of an implant.

7. The tissue product of claim 1, further comprising a second mesh configuration having a second predetermined ratio of a length of each slit to a distance between slits along a long axis of the slits and a second predetermined ration of a width of each slit to the distance of each slit across the long axis of the slits.

8. The tissue product of claim 7, wherein the first predetermined ratio of a length of each slit to a distance between slits along a long axis of the slits is less than about 1:1, or about 1:1, 1.5:1, 2:1, 2.5:1, 3:1, 3.5:1, 4:1, or greater than about 4:1.

9. The tissue product of claim 7, wherein the second predetermined ratio of a width of each slit to the distance between each slit across the long axis of the slits is less than about 1:1, or about 1:1, 1.5:1, 2:1, 2.5:1, 3:1, 3.5:1, 4:1, or greater than about 4:1

10. The tissue product of claim 1, wherein the plurality of slits are oriented orthogonal to a long axis of the matrix.

11. The tissue product of claim 1, wherein the plurality of slits are oriented parallel to a long axis of the matrix.

12. A method of treating tissue, comprising;
selecting a meshed acellular dermal matrix formed as an expandable sheet comprising a plurality of slits having a long axis, the slits extending linearly through a thickness of the sheet and arranged in a pattern forming a mesh configuration;
preparing a recipient site in a body within or contacting a soft tissue; and
securing at least a portion of the meshed acellular dermal matrix to the recipient site.

13. The method of claim 12, wherein the meshed acellular dermal matrix sheet is expanded to cover or retain a tissue implant or anatomic structure.

14. The method of claim 12, wherein the meshed acellular dermal matrix sheet is flexible and curved around an implant.

15. The method of claim 12, further comprising securing at least a portion of a second acellular tissue matrix product to the recipient site.

16. The method of claim 15, wherein the second acellular tissue matrix product is a three-dimensional formed tissue matrix product.

17. The method of claim 12, further comprising implanting a tissue expander to the recipient site.

18. The method of claim 17, wherein the tissue expander comprises a silicone or saline filled implant.

19. The method of claim 12, wherein the meshed acellular dermal matrix has an epithelial basement membrane across the surface.

20. The method of claim 12, wherein the meshed acellular tissue matrix product is anchored using sutures, stables, or other surgical anchors along a lateral border of the pectoralis major muscle extending along an inferior border of the pectoralis major muscle to fix an implant in a stable position.
